(19) United States
(12) Patent Application Publication
(10) Pub. No.: US 2023/0301814 A1
(43) Pub. Date: Sep. 28, 2023

(54) ORTHOPEDIC BRACE HAVING TELESCOPIC LATERAL PANELS AND AN INTERCHANGEABLE WISHBONE COUPLER

(71) Applicant: Aspen Medical Products, LLC, Irvine, CA (US)

(72) Inventors: Jane Price, Anaheim, CA (US); Erik Zimmer, Oceanside, CA (US)

(21) Appl. No.: 18/162,367

(22) Filed: Jan. 31, 2023

Related U.S. Application Data
(60) Provisional application No. 63/305,674, filed on Feb. 1, 2022.
(60) Provisional application No. 63/305,678, filed on Feb. 1, 2022.

(51) Int. Cl. A61F 5/02

(52) U.S. Cl. CPC A61F 5/022

(57) ABSTRACT

An anterior bracing system deployed as part of an orthopedic brace is described. The anterior bracing system features an anterior panel and a plurality of telescopic lateral panels. The anterior panel includes a plurality of rotatable locking members, each disposed on an opposing side of the anterior panel. The telescopic lateral panels are configured to extend in a lateral direction or retract in a medial direction. The plurality of rotatable locking members are configured to rotate between an open position and a closed position. When the locking members are placed in the open position, the telescopic lateral panels are configured to extend or retract. When the locking members are placed the closed position, the telescopic lateral panels are locked in a current position.

### CROSS-REFERENCE TO RELATED APPLICATIONS

This application claims the benefit of priority on U.S. Provisional Application No. 63/305,674 filed Feb. 1, 2022 and U.S. Provisional Application No. 63/305,678 filed Feb. 1, 2022, the entire contents of both of which are incorporated by reference herein.

### FIELD

Embodiments of the disclosure relate to the field of medical devices. More specifically, one embodiment of the disclosure relates to an orthopedic brace and components thereof.

### GENERAL BACKGROUND

The following description includes information that may be useful in understanding the described invention. It is not an admission that any of the information provided herein is prior art or relevant to the presently claimed invention, or that any publication specifically or implicitly referenced is prior art.

Orthopedic braces (orthoses) usually need to be adjusted or customized in some manner to conform to the body part(s) being braced, and then properly positioned. A typical orthosis commonly has at least two portions, a rigid portion supporting a body part, and a flexible or semi-flexible portion securing the orthosis to the body. One type of orthopedic brace is referred to as a “thoracic-lumbar-sacral orthosis” or by the acronym “TLSO.”

A TLSO is a brace that limits movement in a wearer’s spine from the thoracic area (mid-back area) to the wearer’s sacrum (lower-back area). At the same time, the TLSO allows a wearer’s neck to move freely. This type of brace is normally used to provide support and stabilization of the spine after a back injury and/or surgery, and in some cases, may be utilized to address spinal pathologies.

One common problem associated with conventional TLSOs is the donning process is quite difficult, especially when the orthotist is trying to ensure that the TLSO properly fits to immobilize the patient as a proper fit of the orthosis normally provides the wearer with improved pain reduction and promotes healing. Conventional TLSOs rely on soft goods, namely a long belt extending from the posterior brace with a hook and loop fastener that is affixed to a complementary hook and loop fastener located on a top surface of the anterior brace, to secure the anterior brace to the posterior brace. Hence, the donning process may require the patient to be “log rolled” (moved face-down to face-up or vice versa) multiple times in order to adjust and fit the TLSO onto the patient. For example, the patient would be placed onto the posterior bracing system, then “log rolled” onto an anterior brace to allow the belt to be sized with excess belt strap being hidden. Thereafter, the patient may be “log rolled” one or more times to adjust the posterior bracing system or belt adjustment to better immobilize the patient. Avoidance of a complex (and in some cases painful) donning process would ensure more effective usage of TLSO-based orthopedic braces.

Furthermore, there are different braces for different conditions, including the usage of an orthopedic brace with a cervical collar, referred to as Cervical Thoracic Lumbar Sacral Orthosis (CTLSO). It would be advantageous to provide an orthopedic brace that features a uniform construction to enable transitioning from a 3TLSO to a CTLSO and vice versa.

### BRIEF DESCRIPTION OF THE DRAWINGS

Embodiments of the invention are illustrated by way of example and not by way of limitation in the figures of the accompanying drawings, in which like references indicate similar elements and in which:

FIGS. 1A-1D are front perspective views of an exemplary embodiment of an orthopedic brace with an anterior bracing system.

FIG. 2 is a rear perspective view of an exemplary embodiment of the anterior bracing system of FIGS. 1A-1D.

FIG. 3A is a front perspective view of an anterior panel of the anterior bracing system of FIGS. 1A-1D.

FIG. 3B is a rear perspective view of the anterior panel of FIG. 3A illustrating sewing channels on a first side.

FIGS. 4A-4B are rear perspective views of the anterior panel of FIGS. 3A-3B including filler attached thereto.

FIG. 5 is a bottom perspective view of the anterior bracing system of FIGS. 1A-1B

FIGS. 6A-6C are additional front perspective views of various sized anterior panels for embodiments of the anterior bracing system of FIGS. 1A-1D.

FIG. 7A is a front right side perspective view of a first embodiment of a wishbone extension coupler coupling an extension rod and a cervical thoracic orthosis (CTO) clip coupled to a cervical brace.

FIG. 7B is a rear perspective view of the wishbone extension coupler coupling the extension rod and the CTO clip coupled to the cervical brace of FIG. 7A.

FIG. 7C is a side perspective view of the wishbone extension coupler coupling the extension rod and the CTO clip coupled to the cervical brace of FIG. 7A.

FIG. 7D is a front perspective view of a second embodiment of the wishbone extension coupler and its coupling to a CTO clip.

### DETAILED DESCRIPTION

Embodiments of the present disclosure generally relate to an orthopedic brace including an anterior bracing system. According to one embodiment of the disclosure, the anterior bracing system features an orthosis fastening subsystem, which includes telescopic lateral panels (included in an interior chamber between the anterior panel and padding) that extend from opposite sides of the anterior bracing system and are locked in place by locking mechanisms on the opposing sides of the anterior bracing system such that extension of each the telescopic lateral panel assists in configuring an overall sizing of the orthopedic brace when coupled with a posterior bracing system. The orthopedic brace may be placed around a patient’s torso. As described herein, the orthopedic brace may constitute any type of lumbar sacral orthosis (LSO), including a standard LSO (lower back brace belt) or other orthoses configured to provide greater patient immobilization, such as a thoracic LSO (TLSO), or a cervical TLSO (CTLSO) as described below.

When deployed as a TLSO or CTLSO, the anterior bracing system for the orthopedic brace may be configured in accordance with an architecture including an anterior panel having locking mechanisms on opposing sides and telescopic lateral panels slidably coupled to an interior (or “rear”) side of anterior panel. Thus, the anterior bracing system operates such that the telescopic lateral panels are coupled to the anterior panel and slidably extend (or retract) with the locking mechanism of the anterior panel rotating to removably couple with the telescopic lateral panels thereby locking such in a given position. As a result, the telescopic lateral panels may be extended or retracted as a sizing mechanism based on a size of a patient’s torso, e.g., for the purposes of immobilizing the patient (the wearer).

Each of the components of the anterior bracing system may be formed with a rigid material, such as hardened plastic for example, to provide greater stiffness for immobilization of the patient. Herein, the anterior panel is shaped and sized to rest against the mid-to-lower portions of the patient’s front torso to partially immobilize portions of neuromuscular and skeletal systems, e.g., the spine of the patient.

As discussed below and illustrated in the accompanying figures, the interior of the anterior panel includes a plurality of lateral panel guide posts (guide posts) that are configured to couple with guide channels of the telescopic lateral panels. In particular, the combination of the coupling posts of the anterior panel and the guide channels of the telescopic lateral panels enables the extension (or retraction) of the telescopic lateral panels in a controlled manner. Further, locking mechanisms disposed on opposing sides of the exterior of the anterior panel rotate around a hinge and couple with an exterior side of the telescopic lateral panels to lock such in a desired extended or retracted position. The desired extended or retracted position may be in accordance with particular size indicia provided on the exterior side of each telescopic lateral panel.

The lateral panel locking mechanisms (locking mechanism) may each include a hinge and a rotatable locking member that is rotatable around the hinge. The locking mechanisms may be configured to removably lock (or retain) the telescopic lateral panels (lateral panels) in a preferred position through the use of a hook-and-loop fastening system. More particularly, an exterior portion of a lateral panel includes an unbroken loop (UBL) material while a first side of a rotatable locking member includes a hook material to couple with the UBL material such that as the rotatable locking member is rotated and contacts the UBL material, the lateral panel is locked (or retained) in its current position. A second side of the rotatable locking member may have disposed thereon UBL material for coupling with a belt of a posterior bracing system of the orthopedic brace.

The anterior bracing system may also include an extension rod extending vertically upward from the anterior panel. The distal end of the extension rod may have a wishbone extension attachment (wishbone coupler) coupled thereto. The wishbone coupler may further couple with a plurality of attachments thereby providing interchangeability among attachments such as a support chest plate when the orthopedic brace is configured for a TLSO deployment and a CTO clip, which further couples to a cervical brace when the orthopedic brace is configured for a CTLSO deployment.

According to one embodiment, a patient may be positioned on his or her back, e.g., laying on top of a posterior bracing system and the anterior bracing system may be placed on the patient’s front torso such that a liner (padding) rests against the patient. The anterior panel is shaped with a contour intended to conform to the curvature of a front torso region of the patient. The exterior facing surface of the anterior may include two portions of UBL material on opposing sides with each portion at least partially surrounding a locking mechanism. A rotatable locking member of a locking mechanism is rotatable from an open position to a closed position, where in the closed position, the rotatable locking member contacts and couples with the lateral panel causing the lateral panel to be retained in its current position.

Thus, the anterior bracing system may be sized to fit a patient through extension of the lateral panels laterally (outwardly). Upon reaching a desired length of the lateral panel (e.g., an amount a distal edge of the lateral panel has extended laterally from an edge of the anterior panel), the rotatable locking member is rotated from the open position to the closed position and placed in contact with the exterior facing side of the lateral panel causing the lateral panel to be retain in its current position. The lateral panels may lay over a portion of the patient’s side torso. As a result, the length of the lateral panel correlates to the sizing of the anterior bracing system.

The lateral panels extend (or retract) in a substantially perpendicular direction from the patient’s midline. The lateral panels may include one or more slot rails configured to receive guide posts extending in a posterior direction (e.g., toward the patient body) from the anterior panel. Thus, movement of the lateral panels is restricted to be in substantially a perpendicular direction from the patient’s midline.

### I. Terminology

In the following description, certain terminology is used to describe aspects of the invention. For example, the term “member” may be construed as a structural component of an orthopedic brace. In certain situations, a member may include a component covered by soft goods such as one or more textiles, one or more fabrics (woven fabrics and/or non-woven fabrics), leathers, and/or another covering material. These soft goods may feature “loop” type fasteners or other variants to which a “hook” type fastener may be attached or may feature a hook-type fastener for attachment to a loop-type fastener. In other situations, the member may be soft goods attached to another structural component of the orthopedic brace such as a textile or fabric sewn to form together.

The term “chamber” is a partially enclosed housing, namely a structure having partially enclosed perimeter except for one or more openings, such as at least two openings at opposite sides along the periphery. As a result, a partially enclosed chamber operates as a structure that is configured to secure, maintain and protect orthosis fastening subsystem components.

The term “attach” and other tenses of the term (attached, attaching, etc.) may be construed as physically connecting a first member to a second member. A “fastener” may be construed as any physical component that is used to attach different members together. An illustrative example of different types of fasteners and fastening techniques may include, but are not limited or restricted to snaps, buttons, clasps, buckles, adhesives, sewing, heat sealing (or melting), gluing, knitting, or other physical coupling techniques such as a hook and loop connection.

The terms “rigid” or “rigidity” with respect to a member or portion of a member may be construed as the member being configured to at least partially resist bending or deformation. According to this definition, different lengths of a given structure and composition can be rigid at a shorter length, and flexible at a longer length. As used herein, the term “rigid” with respect to a member or portion of a member may be construed as the member could be permanently deformed or broken if bent or twisted by at least 90°. Examples of a rigid member may include, but is not limited or restricted to, an anterior panel, albeit the telescopic lateral panels extended from are rigid, but are flexible to retain a concave-shape without breaking.

Finally, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure is to be considered as an example of the principles of the invention and not intended to limit the invention to the specific embodiments shown and described.

### II. General Architecture

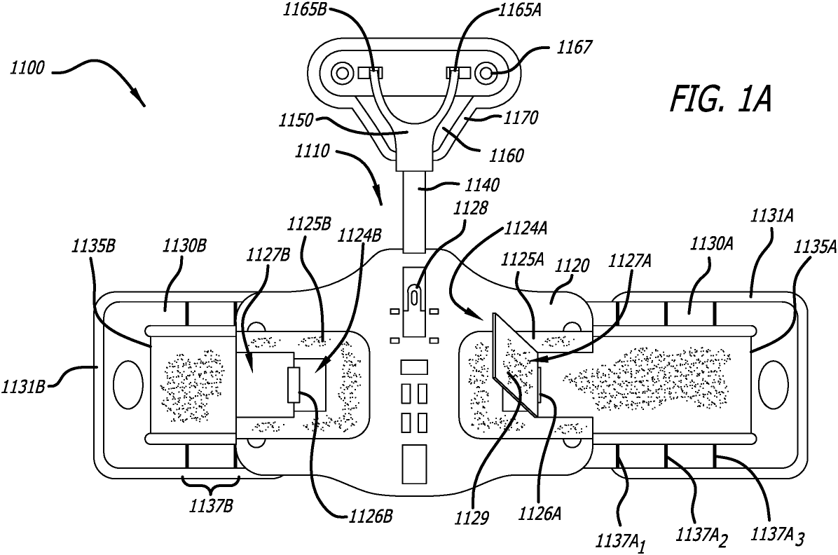

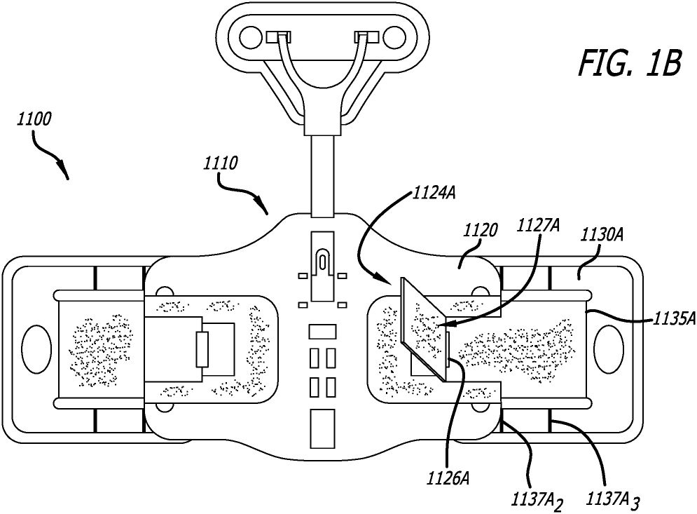

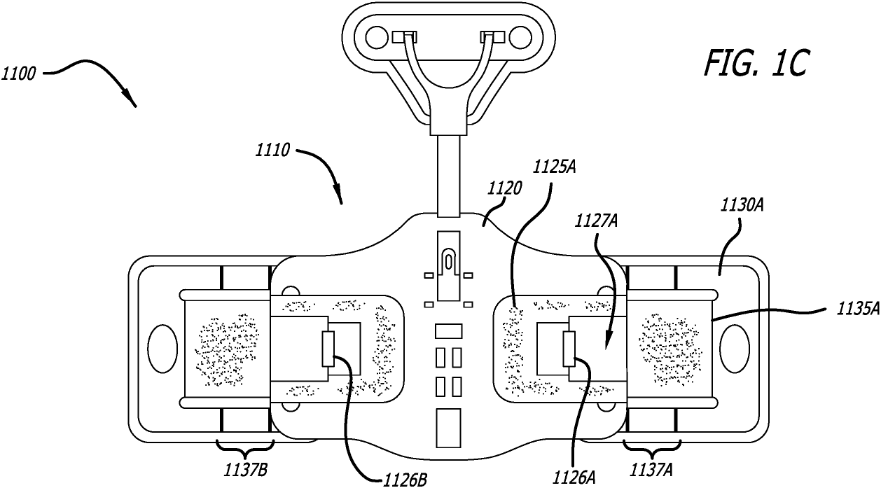

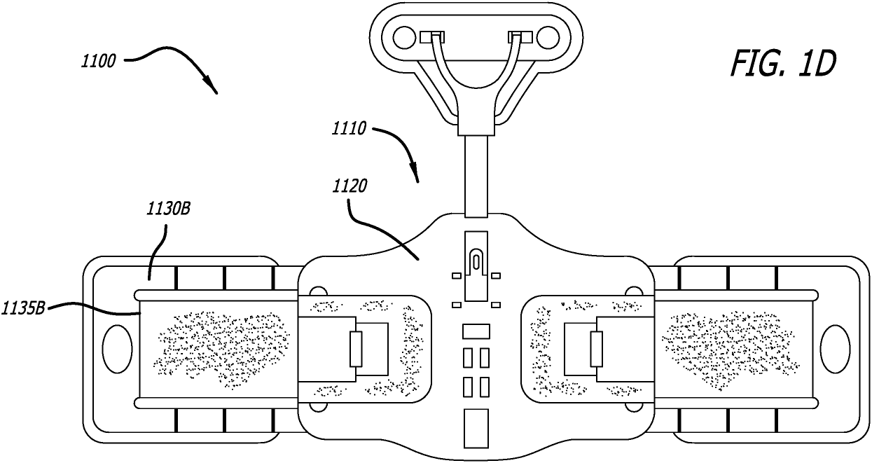

Referring now to FIGS. 1A-1D, a plurality of front perspective views of an exemplary embodiment of an orthopedic brace with an anterior bracing system are shown in accordance with some embodiments. Referring to FIG. 1A, an anterior bracing system 1110 of an orthopedic bracing system 1100 is shown that includes an anterior panel 1120, an extension rod 1140 coupled to the anterior panel 1120 that extends in a superior direction therefrom, and a plurality of lateral panels 1130, such as a first and second telescopic lateral panels 1130A, 1130B that extend laterally therefrom. Herein, the lateral panels 1130 may be made of a rigid or semi-rigid material such as any type of hardened, flexible plastic (e.g., sheet of polyethylene, polycarbonate, etc.). As a result, the lateral panels 1130 may be angularly bent into a concave form to provide lateral support for the orthopedic bracing system 1100.

As shown, the anterior panel 1120 includes an opening receptacle for receiving the extension rod 1140, where an extension rod locking member 1128 rotates between open and closed positions to lock (or retain) the extension rod 1140 in a particular position, where the position of the extension rod 1140 refers to an amount that the extension rod 1140 extends in the superior direction. The anterior panel 1120 also includes a pair of locking mechanisms 1124A, 1124B (collectively, locking mechanisms 1124) disposed on an anterior side, where the locking mechanisms 1124 are configured to engage (couple) with fastener material disposed on an anterior side of the lateral panels 1130.

Further, the locking mechanisms 1124 each include rotatable locking members 1127A, 1127B (collectively, locking members 1127) that are configured to rotate about hinges 1126A, 1126B (collectively, hinges 1126) and fastener portions 1125A, 1125B including a first fastener material (collectively, fastener portions 1125). The locking members 1127 are configured to rotate about their corresponding hinges between an open position and a closed position (or locked position). The closed position refers to the locking member in a position to contact the anterior side of the lateral panels 1130. The open position refers to the locking member being at any position other than the closed position. Further, the locking member includes first and second sides, where a first side includes first fastener material and a second side includes second fastener material. The first side refers to the anterior facing side when the locking member is in the closed position and the second side refers to the posterior facing side when the locking member is in the closed position. Thus, the second side is configured to contact the anterior side of the lateral panels 1130 when the locking member is placed in the closed position.

Further, the terms “first fastener material” and “second fastener material” may refer to complementary components of a hook and loop fastener system such that the first and second fastener materials are configured to couple upon contact.

Additionally, FIG. 1A illustrates that the lateral panels 1130 include filler 1131A, 1131B (collectively, filler 1131 or padding 1131) on a posterior side, with the padding 1131 being configured to rest against the patient body. Additionally, each lateral panel 1130 includes a portion of its anterior side having disposed thereon a second fastener 1135A, 1135B (collectively, first fasteners 1135) that is complementary to the first fasteners 1129 disposed on the rotatable locking members 1127 discussed above. The lateral panels 1130 may also include sizing indicia 1137A₁₋ₓ and 1137B₁₋ₓ (where x>1, and x=3 in FIG. 1A) (collectively, indicia 1137). The indicia 1137 represent predetermined sizing configurations such as large (1137A₁, 1137B₁), medium (1137A₂, 1137B₂), and small (1137A₃, 1137B₃). It is noted that the indicia 1137B_{1-X} are merely represented as 1137B. Thus, the indicia 1137 provide a medical professional sizing parameters that guide the extension or retraction of the lateral panels into a particular sizing configuration. As a result, the sizing indicia 1137 provide the advantage of enabling a medical professional (or wearer) the ability to quickly determine an amount that each lateral panel 1130 should be extended or retracted in order to place the anterior bracing system 1110 into a preconfigured size.

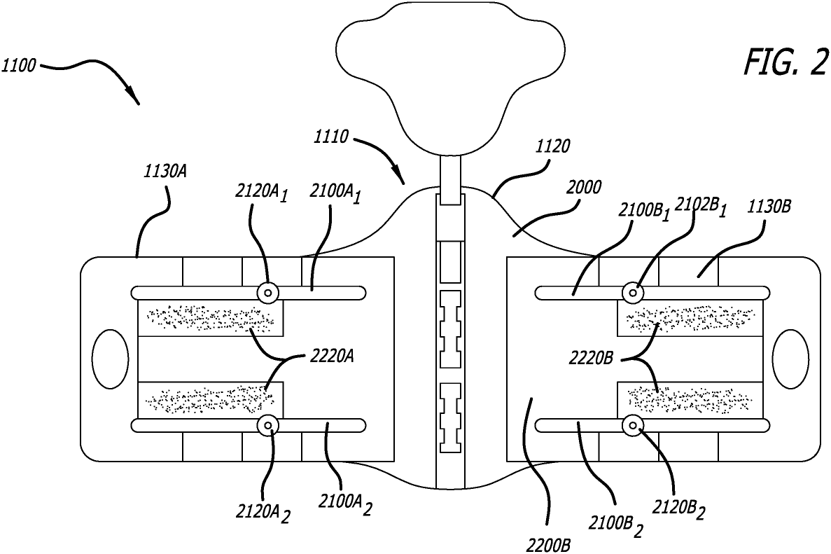

In summary, the anterior bracing system 1110 of FIG. 1A includes an anterior panel 1120 having disposed on an anterior side first and second locking mechanisms that configured to rotate between open and closed positions. Further, the anterior bracing system 1110 includes first and second lateral panels 1130 that are coupled to the posterior side of the anterior panel 1120 via guide posts 2120 (see FIG. 2), with the lateral panels 1130 being configured to extend or retract in the medial/lateral directions. The anterior bracing system 1110 is configured to rest against the front torso of a patient and be sized for the patient through extension or retraction of the lateral panels 1130. Once the lateral panels 1130 have been extended a desired amount based on the size of the patient’s torso, the locking members 1127 are rotated from an open position to a closed position where first fastener material of the lateral panel contacts second fastener material of the locking member causing a coupling therebetween resulting in the lateral panels being locked in the desired position (e.g., desired amount of extension). The retraction and extension of the lateral panels and locking of the same in a desired position are illustrated in FIGS. 1A-1D and will be described further below.

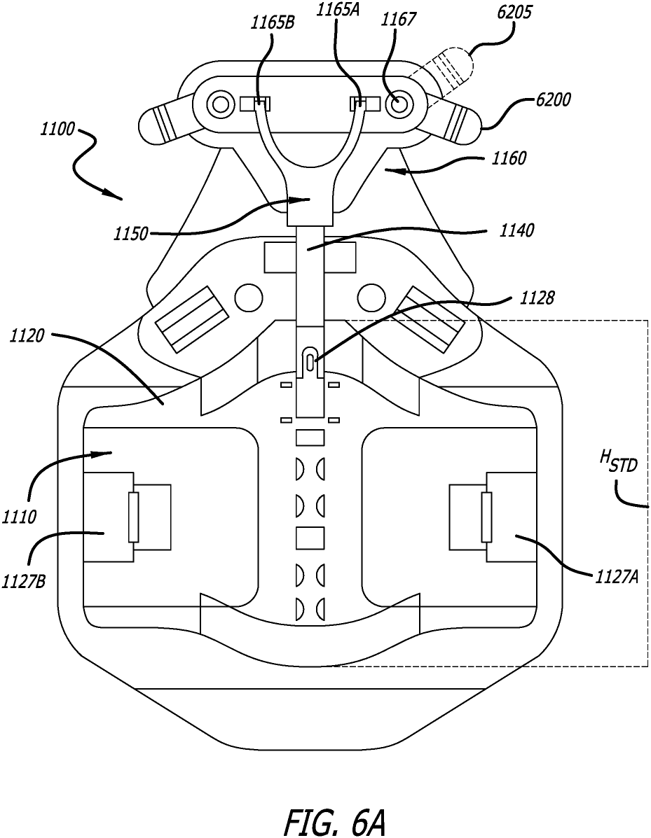

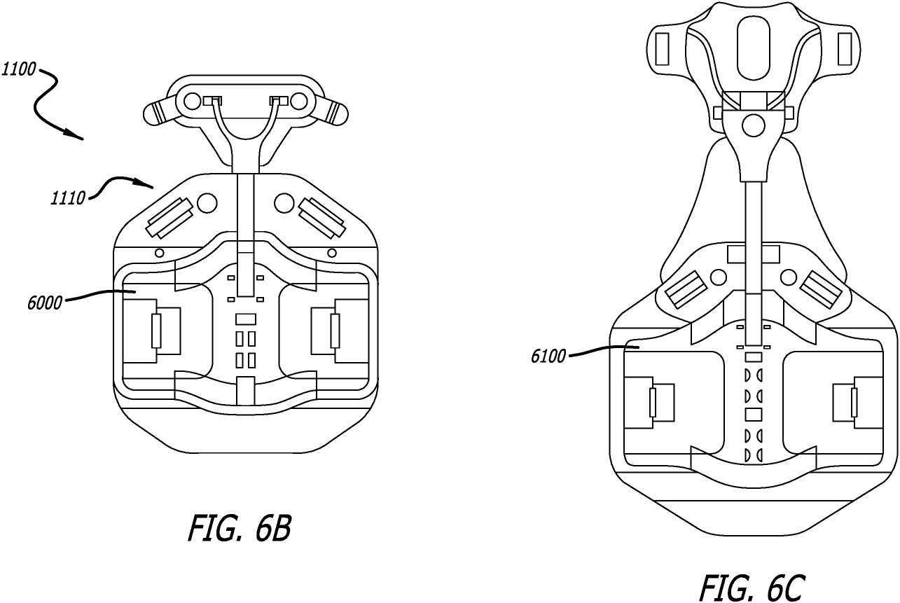

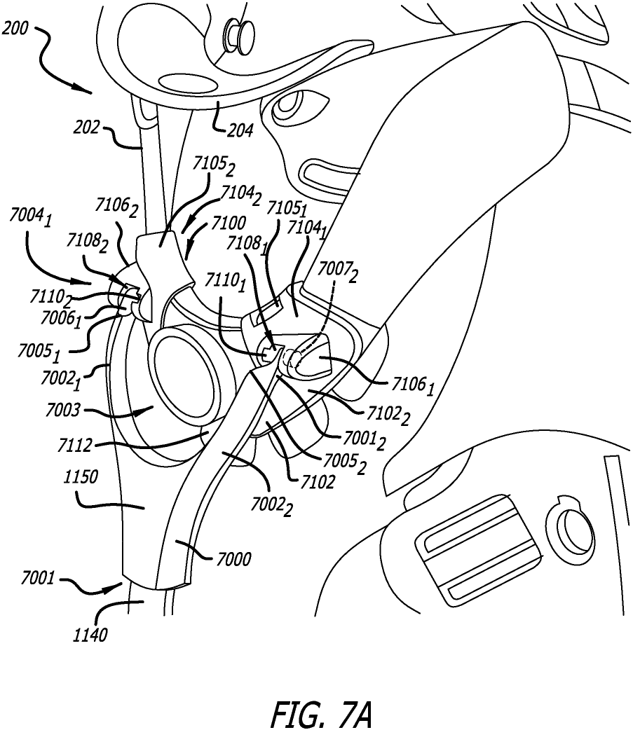

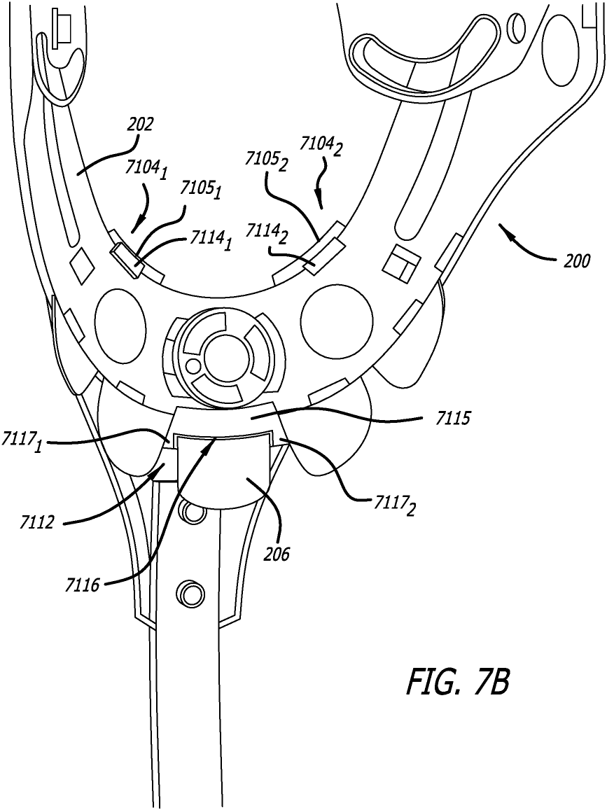

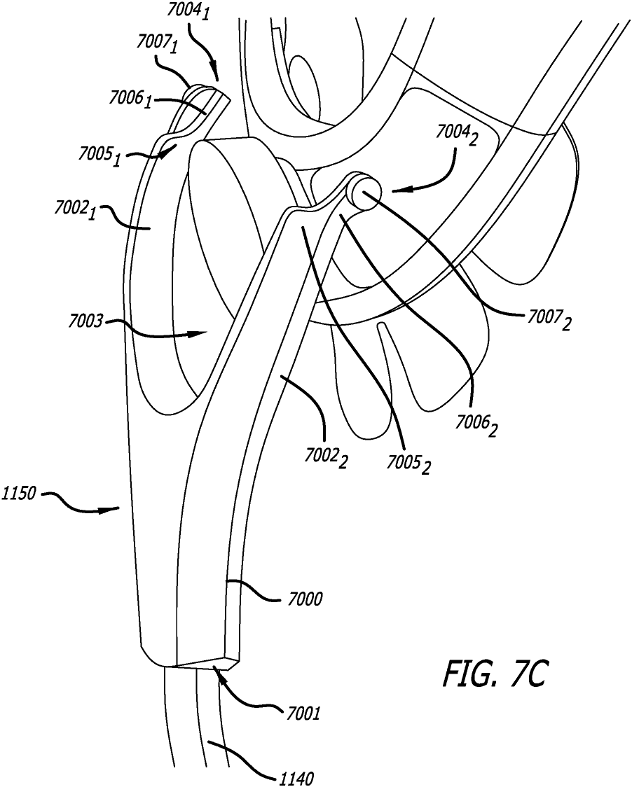

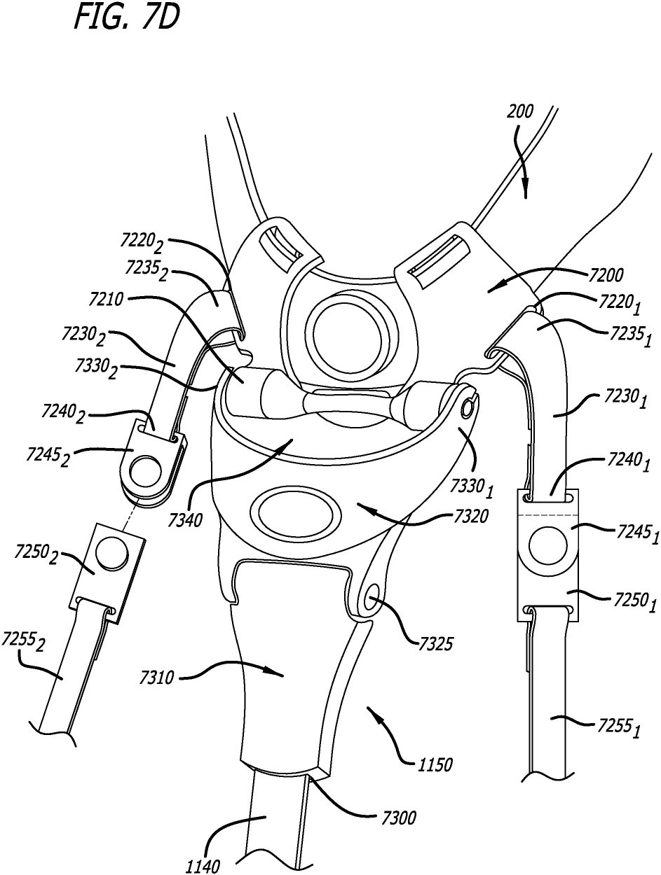

FIG. 1A also illustrates a wishbone extension coupler (wishbone coupler) 1150 coupled to a distal end of the extension rod 1140. The wishbone coupler 1150 is shown as further coupling to a support chest plate 1160 via insertion mechanisms 1165A, 1165B (collectively, insertion mechanisms 1165). The support chest plate 1160 includes snap connectors 1167, which are adapted to receive one or more complementary buckles (see FIG. 6A). As further shown, the support chest plate 1160 features padding 1170 disposed on a posterior side. As discussed below, the wishbone coupler 1150 is configured to permit various attachments to couple with the anterior panel 1120 via the extension rod 1140. As noted above, when the orthopedic brace is configured for a TLSO deployment, the wishbone coupler 1150 couples with the support chest plate 1160 as shown, and when configured for a CTLSO deployment, the wishbone coupler 1150 couples with a cervical brace (see FIGS. 7A-7C).

Referring now to FIG. 1B, the first lateral panel 1130A is shown in a retracted position as compared to FIG. 1A, which illustrates the first lateral panel 1130A in a fully extended position. In particular, the first lateral panel 1130A is retracted (slid in a medial direction) from the fully extended position shown in FIG. 1A to a preconfigured size of “medium” as indicated by the indicia 1137A₂. For instance, the anterior bracing system 1110 may be sized in accordance with a predetermined sizing configuration through the alignment of an outer (lateral) edge 1124A of the anterior panel 1120 with any of the indicia 1137A₁₋ₓ. As shown, the anterior bracing system 1110 is sized according to the indicia 1137A₂ (“medium”). FIG. 1B also illustrates the locking member 1127A in an open position, which allows the lateral panel 1130A to be easily extended or retracted.

Referring now to FIG. 1C, the locking member 1127A is shown in a closed position resulting in a coupling of the fastener materials disposed on the second side of the locking member 1127A and the anterior side of the lateral panel 1130A. This coupling effectively locks the lateral panel 1130A in its current position (e.g., no further extension or retraction is permitted until the locking member and the lateral panel are uncoupled). The locking member 1127A and the lateral panel 1130A may be uncoupled (and again extendible or retractable) through the use of force pulling the locking member 1127A away from the lateral panel 1130A.

Referring now to FIG. 1D, the second lateral panel 1130B is shown in a fully extended position with the locking member 1127B placed in a closed position. Thus, the lateral panel 1130B is locked in its current position until the locking member 1127B and the lateral panel 1130B are uncoupled.

Referring now to FIG. 2, a rear perspective view of an exemplary embodiment of the anterior bracing system of FIGS. 1A-1D is shown in accordance with some embodiments. The rear perspective of FIG. 2 illustrates the posterior side (or interior side) 2000 of the anterior bracing system 1110. As shown, each of the lateral panels 1130A and 1130B includes a set of guide channels 2100A₁₋₂ and 2100B₁₋₂, respectively (collectively, guide channels 2100). Each of the guide channels 2100A₁₋₂ and 2100B₁₋₂ is configured to couple with a corresponding and respective guide post 2120A₁₋₂, 2120B₁₋₂ (collectively, guide post 2120). As shown, the guide posts 2120 are received into the corresponding guide channels 2100, thereby enabling the lateral panels 1130 to slide (extend or retract) along the guide posts 2120.

It is noted that the guide channels 2100 are shown having smooth edges providing for easy, unrestricted or unopposed movement of the lateral panels 1130. However, in some embodiments, the guide channels 2100 may include various ridges or notches that provide some resistance in the movement of the lateral panels 1130. For instance, the edges of the guide channels 2100 may be jagged or include a series of alternative peaks and valleys. In other embodiments, the guide channels 2100 may include either notches or grooves at intervals corresponding to the sizing indicia 1137.

According to one embodiment of the disclosure, the guide posts 2120 may include a columnar body extending in a posterior direction from the anterior panel 1120 such that the columnar body is disposed within a corresponding guide channel 2100. The distal end of the columnar body may include a posterior cap that is sized larger than the width of the guide channels 2100, which operates to maintain the coupling between the lateral panels 1130 and the anterior panel 1120. The posterior cap may take various shapes, which may include a circular cross-section as shown. Further, in assembly, the columnar body may be disposed in an aperture of the anterior panel 1120 and where an anterior end may include an anterior cap that prevents the columnar body from fully extending through the aperture (see FIG. 1D). The lateral panels 1130 may be coupled with the anterior panel 1120 such that the column body of each guide post 2120 are disposed within corresponding guide channels 2100 of the lateral panels. The posterior caps may then be attached to each guide post 2120 (e.g., via a screw fastener or adhesive).

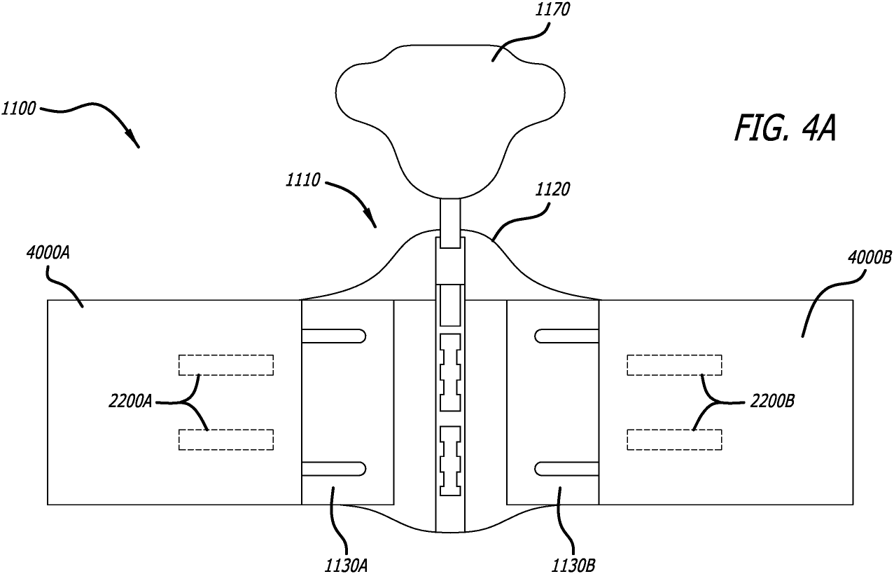

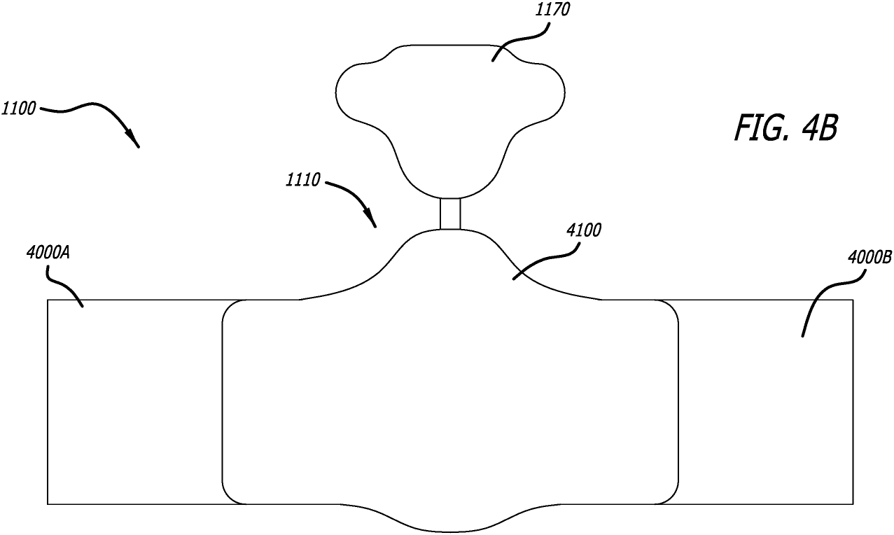

Additionally, the anterior side of each lateral panel 1130 may include one or more strips of fastener material 2220A, 2220B that is configured to couple with corresponding fastener material disposed on filler 4000A, 4000B (see FIGS. 4A-4B) or couple directly with the anterior side of the filler 4000A, 4000B.

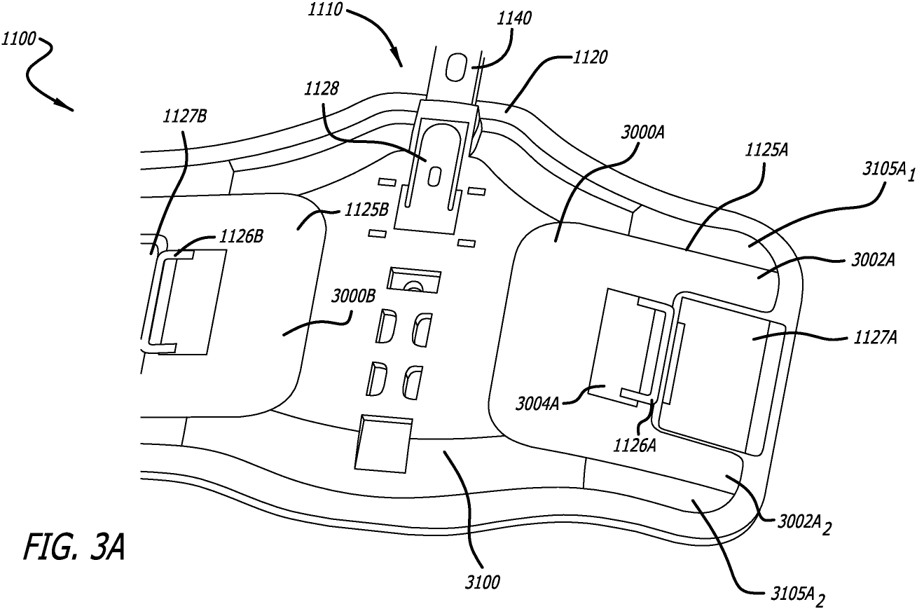

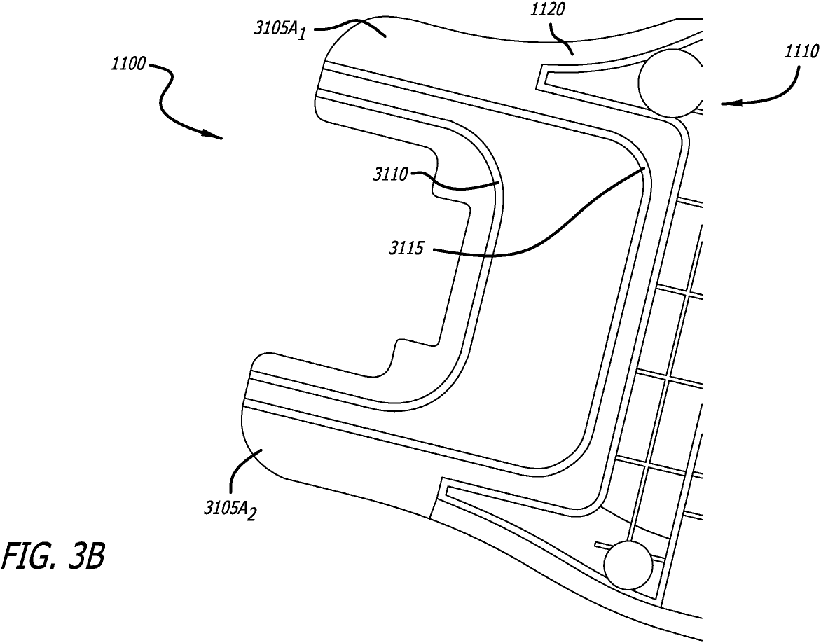

Referring now to FIG. 3A, a front perspective view of the anterior panel 1120 of the anterior bracing system 1110 of FIGS. 1A-1D is shown in accordance with some embodiments. The anterior bracing system 1110 illustrates the anterior panel 1120 without the lateral panels 1130 or the extension rod 1140 coupled thereto. Further, the anterior panel 1120 is illustrated without the fastener material overlays that are illustrated in FIGS. 1A-2. Thus, FIG. 3A provides a clear illustration of the locking mechanisms 1124, especially the components of the locking mechanism 1124A. In particular, the locking member 1127A is shown in a closed position and the hinge 1126A around which the locking member 1127A rotates. Additionally, the hinge attachment portion 3004A is shown, which couples the hinge 1126A to the anterior panel 1120 and is disposed under the fastener material overlay of FIGS. 1A-2.

The outline of the fastener portions 1125 are shown such that the fastener portion 1125A includes a base portion 3000A and two portions 3002A₁₋₂ that extend laterally from the base portion 3000A and partially surround the locking member 1127A when such is in the closed position. In some embodiments, the anterior panel 1120 may be segmented into a middle body portion 3100 and lateral portions 3105A₁₋₂, 3105B₁₋₂ that are disposed laterally to the middle body portion 3100. For instance, the two portions 3002A₁₋ ₂ of the fastener portion 1125A may be disposed on the lateral portions 3105A₁₋₂. Similarly, although not shown, the two portions 3002B₁₋₂ of the fastener portion 1125B may be disposed on the lateral portions 3105B₁₋₂.

Referring to FIG. 3B, a rear perspective view of the anterior panel 1120 of FIG. 3A illustrating sewing channels on a first side is shown in accordance with some embodiments. The rear perspective of FIG. 3B illustrates the anterior side of the anterior panel 1120 and depicts one embodiment of sewing channels 3110, 3115. In some embodiments, the fastener portion 1125A may be sewn to the anterior panel 1120 along the sewing channels 3110, 3115. An adhesive may be used in addition in some embodiments as well as an alternative in others.

Referring to FIGS. 4A-4B, rear perspective views of the anterior panel of FIGS. 3A-3B including filler attached thereto are shown in accordance with some embodiments. FIG. 4A illustrates that filler (padding) 4000A, 4000B may be placed on an anterior side of the lateral panels 1130 and be configured to rest against the patient thereby providing cushioning between the lateral panels 1130A, 1130B and the patient’s body. The padding 4000A, 4000B may couple with the anterior side of the lateral panels 1130 via one or more strips of fastener material 2220A, 2220B. In some embodiments, the anterior side of the padding 4000A, 4000B includes complementary fastener material strips (not shown). In other embodiments, as shown, the posterior side of the padding 4000A, 4000B couples directly to the strips of fastener material 2220A, 2220B. In other embodiments, various fastener means may be utilized such as snap fasteners and/or buttons (wherein each of the anterior panel and the padding 4000A, 4000B would include a complementary component of a snap fastener and a button fastening mechanism (e.g., a button and a receptacle)). FIG. 4B illustrates that a center torso padding 4100 may also be placed between the patient’s body and the anterior bracing system 1110. Although not shown, the padding 4100 may also be coupled to the anterior side of the anterior panel with one or more fastener material strips, snap fasteners and/or buttons.

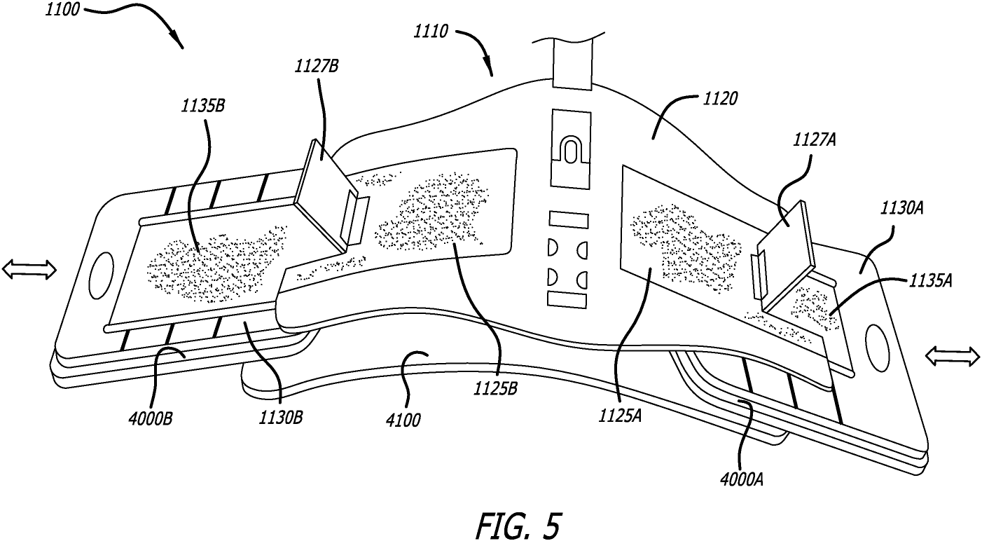

Referring to FIG. 5, a bottom perspective view of the anterior bracing system 1110 of FIGS. 1A-1B is shown in accordance with some embodiments. In particular, FIG. 5 illustrates that the lateral panels 1130 may extend or retract such that movement occurs between the anterior panel 1120 and the padding 4100. Further, FIG. 5 provides a clear illustration of each of the locking members 1127, namely locking member 1127A and locking member 1127B, placed in an open position. When in the open position, the locking members 1127 resemble flaps that extend outwardly from the anterior panel 1120.

Referring to FIGS. 6A-6C, additional front perspective views of various sized anterior panels for embodiments of the anterior bracing system of FIGS. 1A-1D are shown in accordance with some embodiments. The variations in the sizing of the anterior panels of FIGS. 6A-6C highlight one aspect of the interoperability / interchangeability of the anterior bracing system 1100 disclosed herein. In particular, the anterior panel 1120 of FIG. 6A (and also illustrated in several of FIGS. 1A-5) may be referred to as a standard-sized anterior panel, which is indicated as having a first height (H_{STD}). In contrast, the anterior panel 6000 of FIG. 6B may be referred to as a petite-sized anterior panel having a second height that is less than H_{STD} and the anterior panel 6100 of FIG. 6C may be referred to as a grande-sized anterior panel having a third height that is greater than H_{STD}.

Although the size of the anterior panels 1120, 6000, 6100 may differ, each maintains the same connectivity with the lateral panels 1130 and the extension rod 1140. In some instances, a shortened extension rod may be used with the anterior panel 6000 as compared to the extension rod 1140. Both the extension rod 1140 and any shortened (or lengthened) version may couple with any of the anterior panels 1120, 6000, 6100 as well as with the wishbone coupler 1150. Also, a single buckle 6200 or multiple (two or more) buckles 6200, 6205, which are positioned on a distal end of support straps received from the posterior bracing system, may be connected simultaneously to each snap connector 1167 on the support chest plate 1160.

Thus, a medical professional may easily swap out one anterior panel 1120, 6000, 6100 for another version depending on the size of the patient while utilizing the same components otherwise. In some instances, a shortened extension rod may be used in place of the extension rod 1140.

### III. Wishbone Coupler and Cervical Thoracic Orthosis Clip

In FIGS. 1A-6C, an anterior bracing system 1100 was illustrated and described detailing the inclusion of an anterior panel and telescopic lateral panels as well as the operability of the telescopic lateral panels to extend or retract in a lateral or medial direction and be locked into place through the transitioning of locking mechanisms from an open state to a closed state. As a result, the telescopic lateral panels may be locked in a particular sizing configuration. Additionally, many of the FIGS. 1A-6C included an extension rod extending in a superior distal direction from the anterior panel where the distal end of the extension includes a wishbone coupler that was coupled to either a support chest plate or a cervical thoracic orthosis (CTO) clip. FIGS. 7A-7C and the ensuing description detail aspects of the wishbone coupler and the coupling to a CTO clip. However, the coupling of the wishbone coupler to the CTO clip operates in the same manner as coupling the wishbone coupler to a support chest plate (see FIGS. 1A-1D); thus, the discussion below pertaining to the coupling of the wishbone coupler to the CTO clip applies equally to the coupling of the wishbone coupler to the support chest plate.

Referring now to FIG. 7A, a front right side perspective view of a first embodiment of the wishbone coupler 1150 coupling the extension rod 1140 and a cervical thoracic orthosis (CTO) clip 7100 coupled to a cervical brace is shown in accordance with some embodiments. A distal end of the extension rod 1140 is shown coupled with the wishbone coupler 1150, which is rotatably coupled to the CTO clip 7100. The CTO clip 7100 is, in turn, coupled to a cervical collar 200 (e.g., a cervical brace), and specifically to a chest support brace 202. The cervical collar 200 further comprises a front cervical collar portion 204.

The extension rod 1140 may be coupled to the wishbone coupler 1150 through insertion of a distal end of the extension rod 1140 into a receiving cavity 7001 positioned at a bottom (inferior) end of the wishbone coupler 1150. The extension rod 1140 may be retained within the receiving cavity 7001 through interconnections of one or more latches or notches (not shown).

The wishbone coupler 1150 includes a body portion 7000, the receiving cavity 7001, and extension arms 7002₁₋₂ (collectively, extension arms 7002) extending distally in a superior direction from the body portion 7000. As shown, the extension arms 7002 form a wishbone shape in which the extension arms 7002 each extend distally in the superior direction at slightly opposing angles relative to the midline of the wishbone coupler 1150. This architecture has the effect of creating an opening 7003 between the extension arms 7002. The extension arms 7002 terminate at distal ends 7004₁₋₂ (collectively, distal ends 7004) and also include distal ridges 7005₁₋₂ (collectively, distal ridges 7005) as well as distal connection limbs 7006₁₋₂ (collectively, connection limbs 7006), where connection knobs 7007₁₋₂ (collectively, connection knobs 7007) are disposed at the terminal end of the distal connection limbs 7006₁₋₂ (see FIG. 7C).

In coupling the wishbone coupler 1150 to the CTO clip 7100, the distal connection limbs 7006 extend into the receiving components 7106₁₋₂ of the CTO clip 7100. FIG. 7A illustrates an anterior side of the CTO clip 7100, which includes a body portion 7102 formed from first and second extensions 7104₁₋₂ (collectively, first and second extensions 7104) and a lower support portion 7112. The first and second extensions 7104 include the first and second receiving components 7106₁₋₂ (collectively, receiving components 7106), which are configured to receive the connection limbs 7006 and house the connection knobs 7007. The receiving components 7106 include cavities 7108₁₋₂ (collectively, cavities 7108) and slots 7110₁₋₂ (collectively, slots 7110).

The CTO clip 7100 also includes upper ridges 7105₁₋₂ (collectively, upper ridges 7105) which couple with an upper portion of the chest support brace 202 of the cervical collar 200, with is also shown in FIG. 7B. Referring to FIG. 7B, a rear perspective view of the wishbone extension coupler coupling the extension rod and the CTO clip coupled to the cervical brace of FIG. 7A is shown in accordance with some embodiments. Posterior notches 7114₁₋₂ extend from the upper ridges 7105 and grip a posterior side of the chest support brace 202. Additionally, a lower posterior clip 7115 of the CTO clip 7100 is shown where lower bridge portions 7117₁₋₂ couple the lower posterior clip 7715 to the anterior side 7113 of the CTO clip 7100 as shown in FIG. 7A. An aperture 7116 may be formed between the lower bridge portions 7117₁₋₂ and be configured to receive a tongue portion 206 of the CTO clip 7100.

Referring to FIG. 7C, a side perspective view of the wishbone extension coupler coupling the extension rod and the CTO clip coupled to the cervical brace of FIG. 7A is shown in accordance with some embodiments. FIG. 7C provides an illustration of the wishbone coupler 1150 adjacent to the cervical brace (cervical collar) 200. As noted above, FIG. 7C provides an illustration of the distal ends 7004 of the extension arms 7002, which include the distal ridges 7005, the connection limbs 7006, and the connection knobs 7007.

Referring now to FIG. 7D, a perspective view of a second embodiment of the wishbone coupler 1150 coupling the extension rod 1140 and a cervical thoracic orthosis (CTO) clip 7200 coupled to a cervical brace is shown in accordance with some embodiments. A distal end of the extension rod 1140 is shown coupled with the wishbone coupler 1150, which is rotatably coupled to the CTO clip 7200. The CTO clip 7200 is, in turn, coupled to a cervical collar 200 (e.g., a cervical brace).

The extension rod 1140 may be coupled to the wishbone coupler 1150 through insertion of a distal end of the extension rod 1140 into a receiving cavity 7300 positioned at a bottom (inferior) end of the wishbone coupler 1150. The extension rod 1140 may be retained within the receiving cavity 7300 through interconnections of one or more latches or notches (not shown).

The wishbone coupler 1150 includes a first body portion 7310, a second body portion 7320 rotationally coupled to the first body portion 7310 by hinge 7325 oriented with a substantially horizontal rotational axis, and extension arms 7330₁₋₂ (collectively, extension arms 7330) extending distally in a superior direction from the second body portion 7320. As shown, the extension arms 7330 form a wishbone shape in which the extension arms 7330 each extend distally in the superior direction at slightly opposing angles relative to the midline of the wishbone coupler 1150. This architecture has the effect of creating an opening 7340 between the extension arms 7330. The extension arms 7330 terminate at distal ends 7335₁₋₂ (collectively, distal ends 7335), which is adapted to be rotationally coupled to a coupling member 7210 of the CTO clip 7200.

For coupling the wishbone coupler 1150, the CTO clip 7200 also includes a first set of fastening members 7220₁₋₂ (collectively, fastening members 7220). As an illustrative example, the fastening members 7220 may constitute “D-ring” fasteners (single or double) that are integrated as part of the CTO clip 7200. The D-ring fasteners 7220₁₋₂ are configured to receive first support straps 7230₁₋₂, where each of the first support straps 7230₁₋₂ includes a first end portion 7235₁₋₂ and a second end portion 7240₁₋₂. More specifically, the first end portion 7235₁ of the first support strap 7230₁ is securely attached to the D-ring fastener 7220₁. This attachment may be accomplished though complementary hook and loop fasteners (UBL material) positioned along opposite surfaces at the first end portion 7235₁ after insertion through the D-ring fastener 7220₁. A similar arrangement may occur between the D-ring fastener 7220₂ and the second end portion 7235₂ of the first support straps 7230₂.

As further shown in FIG. 7D, a distal part of each of the second end portions 7240₁₋₂ includes a bracket 7245₁₋₂, which is to be fastened to a complementary bracket 7250₁₋₂ oriented on a distal end of second support strap 7255₁₋₂, respectively. More specifically, the bracket 7245₁ of the first support strap 7230₁ is securely attached to the complementary bracket 7250₁ of the second support strap 7255₁. Similarly, the bracket 7245₂ of the second support strap 7230₂ is securely attached to the complementary bracket 7250₂ of the second support strap 7255₂. The support straps 7255₁₋₂ may extend under the axilla of the wearer.

In the foregoing description, the invention is described with reference to specific exemplary embodiments thereof. Hence, it will be evident that certain components may be deployed within different types of orthopedic devices and various modifications and changes may be made thereto without departing from the broader spirit and scope of the invention as set forth in the appended claims.

What is claimed is:

1. An anterior bracing system deployed as part of an orthopedic brace, comprising:
an anterior panel including a plurality of rotatable locking members each disposed on an opposing side of the anterior panel; and
a plurality of telescopic lateral panels configured to extend in a lateral direction or retract in a medial direction, wherein the plurality of telescopic lateral panels are disposed on a posterior side of the anterior panel,
wherein the plurality of rotatable locking members are configured to rotate between an open position and a closed position such that when in the open position, the plurality of telescopic lateral panels are configured to extend or retract and when in the closed position, the plurality of telescopic lateral panels are locked in a current position.

2. The anterior bracing system of claim 1, wherein at least a first telescopic lateral panel of the plurality of telescopic lateral panels includes an anterior surface with a first fastener disposed thereon, the first fastener is complementary to a second fastener disposed on a posterior side of a first rotatable locking member of the plurality of rotatable locking members.

3. The anterior bracing system of claim 2, wherein the first fastener disposed on the first telescopic lateral panel includes a first unbroken loop (UBL) material while the second fastener disposed on the posterior side of the first rotatable locking member includes a hook material configured for coupling with the UBL material.

4. The anterior bracing system of claim 3, wherein an anterior side of the first rotatable locking member includes a second UBL material for coupling with a belt of a posterior bracing system.

5. The anterior bracing system of claim 3, wherein each of the plurality of telescopic lateral panels includes a set of guide channels oriented in a lateral and medial direction, a first set of guide channels is configured to receive and retain a corresponding guide post coupled to a first telescopic lateral panel of the plurality telescopic lateral panels, thereby enabling the first telescopic lateral panel to extend or retract.

6. The anterior bracing system of claim 3, wherein the anterior panel is segmented into a middle body portion and a pair of lateral portions that are disposed laterally to the middle body portion.

7. The anterior bracing system of claim 1 further comprising one or more sewing channels, the first fastener is be sewn to the anterior panel along the sewing channels.

8. The anterior bracing system of claim 1, wherein the anterior panel further includes (i) an opening receptacle adapted to receive an extension rod and (ii) an extension rod locking member adapted to rotate between an open position that allows for a release or insertion of the extension rod and a closed position to lock and retain the extension rod in a particular position.

9. The anterior bracing system of claim 8, wherein a first distal end of the extension rod includes a wishbone coupler, the wishbone coupler includes a body portion and a pair of extension arms extends in a superior direction and at slightly opposing angles relative to a midline of the wishbone coupler.

10. The anterior bracing system of claim 9, wherein the pair of extension arms of the wishbone coupler are coupled to a cervical thoracic orthosis (CTO) clip that is securely fastened to a cervical brace supported by the wishbone coupler.

11. The anterior bracing system of claim 9, wherein the CTO clip includes at least a first fastening member and a second fastening member, both of the first fastening member and the second fastening member are configured to receive support straps coupled to a posterior panel to be positioned on an opposite side of the wearer.

12. The anterior bracing system of claim 9, wherein the pair of extension arms of the wishbone coupler are coupled to a support chest plate.

13. The anterior bracing system of claim 1, wherein the anterior panel includes a middle body portion includes a first pair of lateral portions extending in a first direction from the middle body portion and a second pair of lateral portions extending in a second direction from the middle body portion opposite to the first direction, the first pair of lateral portions partially surrounding a first rotatable locking member of the plurality of rotatable locking members and the second pair of lateral portions partially surrounding a second rotatable locking member of the plurality of rotatable locking members.

14. An anterior bracing system deployed as part of an orthopedic brace, comprising:
an anterior panel including a plurality of rotatable locking members each disposed on an opposing side of the anterior panel; and
a plurality of telescopic lateral panels configured to extend in a lateral direction or retract in a medial direction,
wherein each of the plurality of rotatable locking members is configured to rotate between an open position in which a telescopic lateral panel of the plurality of telescopic lateral panels is placed into a selected extended orientation and a closed position in which the telescopic lateral panel is locked into the selected extended orientation.

15. The anterior bracing system of claim 14, wherein at least a first telescopic lateral panel of the plurality of telescopic lateral panels includes an anterior surface with a first fastener disposed thereon, the first fastener is complementary to a second fastener disposed on a posterior side of a first rotatable locking member of the plurality of rotatable locking members.

16. The anterior bracing system of claim 15, wherein the first fastener disposed on the first telescopic lateral panel includes a first unbroken loop (UBL) material while the second fastener disposed on the posterior side of the first rotatable locking member includes a hook material configured for coupling with the UBL material.

17. The anterior bracing system of claim 16, wherein an anterior side of the first rotatable locking member includes a second UBL material for coupling with a belt of a posterior bracing system.

18. The anterior bracing system of claim 14, wherein each of the plurality of telescopic lateral panels includes a set of guide channels oriented in a lateral and medial direction, a first set of guide channels is configured to receive and retain a corresponding guide post coupled to a first telescopic lateral panel of the plurality telescopic lateral panels, thereby enabling the first telescopic lateral panel to extend or retract.

19. The anterior bracing system of claim 14, wherein the anterior panel further includes (i) an opening receptacle adapted to receive an extension rod and (ii) an extension rod locking member adapted to rotate between an open position that allows for a release or insertion of the extension rod and a closed position to lock and retain the extension rod in a particular position.

20. The anterior bracing system of claim 19, wherein a first distal end of the extension rod includes a wishbone coupler, the wishbone coupler includes a body portion and a pair of extension arms extends in a superior direction and at slightly opposing angles relative to a midline of the wishbone coupler.

21. The anterior bracing system of claim 20, wherein the pair of extension arms of the wishbone coupler are coupled to a cervical thoracic orthosis (CTO) clip that is securely fastened to a cervical brace.

22. The anterior bracing system of claim 20, wherein the pair of extension arms of the wishbone coupler are coupled to a support chest plate.

23. An anterior bracing system, comprising:
an anterior panel;
a padding; and
a plurality of telescopic lateral panels configured to extend in a lateral direction or retract in a medial direction from an interior chamber formed between the anterior panel and the padding,
wherein each telescopic lateral panel of the plurality of telescopic lateral panels is placed into a selected extended orientation and locked to maintain in the selected extended orientation.
